(19) United States
(12) Patent Application Publication
(10) Pub. No.: US 2007/0142786 A1
(43) Pub. Date: Jun. 21, 2007

(54) System with color-coded medical syringes and basins

(76) Inventors: Fred P. Lampropoulos, Salt Lake City, UT (US); Larry Tolman, Alpine, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 11/305,358

(22) Filed: Dec. 16, 2005

(51) Int. Cl. A61M 5/00

(52) U.S. Cl. 604/189

(57) ABSTRACT

A medical fluid delivery system having color coding or other non-textual indicia which associates a medicinal fluid delivery device with a corresponding medicinal fluid. The medicinal fluid delivery system comprises a medicinal fluid delivery device, a medicinal fluid container, and non-textual indicia. A non-textual indicium is integrated in the medicinal fluid delivery device, and a corresponding non-textual indicium is integrated in the medicinal fluid container. The non-textual indicium associates the delivery device with the container, and hence the medicinal fluid contained therein. The non-textual indicium can be the color of the medicinal fluid delivery device, and the corresponding non-textual indicium can be the color of the medicinal fluid container. The non-textual indicium can include a chemical indicator strip having an exposed reactant surface configured to display a particular color when exposed to a medicinal fluid.

### BACKGROUND OF THE INVENTION

1. The Field of the Invention

The present invention relates to a medicinal fluid delivery system. More particularly, the present invention relates to a medicinal fluid delivery system having non-textual indicia to quickly identify a medicinal fluid container for filling an associated medicinal fluid delivery device with a desired medicinal fluid.

2. The Relevant Technology

Medical syringes play a vital role in the treatment and care of patients in medical environments. Medical syringes provide a quick, easy and relatively unobtrusive means for administering medicinal fluids to a patient's body during a medical procedure.

In many medical procedures, several different medicinal fluids are utilized in connection with the care and treatment of a patient. Some medicinal fluids have similar remedial and/or chemical properties, whereas others can have quite different remedial effects. In some cases the use and/or mixing of medicinal fluids have divergent qualities that can be dangerous to the patient. During a particular medical procedure where multiple medicinal fluids, containers and medical syringes are utilized, a practitioner can become confused or unsure as to what medicinal fluid is contained in a particular medical syringe. For example, in some medical procedures multiple medicinal fluids are provided in a medical tray. Multiple containers, such as medicine cups or basins, are placed on the medical tray. The containers are then filled with a particular medicinal fluid so that the practitioner has quick and easy access to the medicinal fluids. During the course of the procedure the practitioner administers the various medicinal fluids to the patient. Where the practitioner empties all the medicinal fluid from one of the medical syringes and thereafter seeks to reload the same syringe with the same medicinal fluid, the practitioner can become confused as to the original contents of the syringe. In addition, during more complex procedures a practitioner may become confused as to what medicinal fluid is contained within a syringe.

One approach to resolve this confusion is to print a textual indication on the outside surface of the barrel of the syringe to indicate the medicinal fluid contained therein. While providing a textual indication of the contents of the syringe can be helpful to identify the fluid contained in the syringe, the textual indication does not resolve the problems associated with proper identification of syringes and associated medicinal fluids. For example, in complex or time sensitive procedures, the step of reading the textual indications may be overlooked or impractical due to the exigencies of the procedure. Additionally, the practitioner is forced to assume that a correct fluid has been loaded or reloaded into the syringe and that the syringe does in fact hold the indicated fluid. In addition, the textual indication may not facilitate reloading a particular syringe from a corresponding container holding the desired medicinal fluid.

### BRIEF SUMMARY OF THE INVENTION

The present invention is directed to a medicinal fluid delivery system having color coding or other non-textual indicia that serve to associate a medicinal fluid delivery device with the corresponding medicinal fluid. The non-textual indicia allow a practitioner to quickly and effectively associate a medicinal fluid delivery device with the medicinal fluid container having the corresponding medicinal fluid. The present invention is helpful in surgical environments where multiple medicinal fluids, medicinal fluid containers, and medicinal fluid delivery devices are utilized during the course of a surgical procedure. The present invention helps to minimize the potential for error associated with the delivery of medicinal fluids by providing a mechanism to quickly and effectively associate a medicinal fluid delivery device with the corresponding medicinal fluid and medicinal fluid container.

In one exemplary embodiment, a medicinal fluid delivery system comprises a medicinal fluid delivery device, a medicinal fluid container, and color coding or other non-textual indicia to associate the medicinal fluid delivery device with the medicinal fluid container and the medicinal fluid contained therein. The medicinal fluid delivery device can comprise a medical syringe having a color coded plunger rod which is matched to the color of a medicinal fluid container, basin, or medicinal bottle. The color of the medical syringe and the corresponding color of the medicine cup allow a practitioner to quickly and effectively associate the medical syringe with the medicinal fluid contained in the associated medicine cup. In one embodiment, the color of the plunger rod and the medicinal fluid container is indicative of the fluid utilized therewith. In another embodiment, a different component of the medicinal fluid delivery device is color coded and only a portion of the medicine cup is the color coded.

In an alternative embodiment of the present invention, non-textual indicia comprise an indicator mechanism such as chemical indicator strips having an exposed reactant surface. The exposed reactant surface is configured to display a particular color when a medicinal fluid contacts the exposed surface. This allows a user to quickly look at the color on the indicator strip of the medicinal fluid delivery device and the color on the indicator strip of the medicinal fluid container to determine whether they contain the corresponding medicinal fluid. In one embodiment, a chemical indicator strip is embedded in the barrel of the syringe and an associated chemical indicator strip is embedded in an inner wall of the medicine cup. In another embodiment, the chemical indicator strip is embedded in an attachment member of the syringe, such as a luer coupler or stop cock.

In one embodiment, the medicinal fluid delivery device comprises a control syringe linked to a manifold having two or more input ports. In the embodiment, the first input port includes a color that corresponds to the color of an associated medicinal fluid container. The medicinal fluid container is a first medicinal syringe that includes a color corresponding to the color of the first input port. The color of the first input port and the corresponding color of the medical syringe facilitate quick and effective association between the first input port and the medicinal fluid contained within the first medical syringe.

These and other objects and features of the present invention will become more fully apparent from the following description, or may be learned by the practice of the invention as set forth hereinafter.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and: explained with additional specificity and detail through the use of the accompanying drawings in which:

FIG. 1 is a perspective view of a medicinal fluid delivery system illustrating a medicinal fluid delivery device and medicinal fluid container having non-textual indicium;

FIG. 2 is a perspective view of a medicinal fluid delivery system having a plurality of color coded medicinal fluid delivery devices and medicinal fluid containers;

FIG. 3 is a perspective view of a medicinal fluid delivery system illustrating loading of a medicinal fluid into a medicinal fluid delivery device from a corresponding medicinal fluid container;

FIG. 4 is a perspective view of a medicinal fluid delivery system having a manifold system according to an alternative embodiment of the present invention;

FIG. 5A is a perspective view of a medicinal fluid delivery system having an indicator mechanism according to an alternative embodiment of the present invention; and

FIG. 5B is a perspective view of a medicinal fluid delivery system having non-textual indicia on an attachment member of the medicinal fluid delivery device.

### DETAILED DESCRIPTION OF THE PREFERRED EMBODIMENTS

The present invention is directed to a medicinal fluid delivery system having color coding or other non-textual indicia that serve to associate a medicinal fluid delivery device with the corresponding medicinal fluid. According to one exemplary embodiment, the medical fluid delivery system includes color coded components or other non-textual indicia that serve to associate a medicinal fluid delivery device with a medicinal fluid container. A color coded component or other non-textual indicium is integrated in the medicinal fluid delivery device, and a corresponding indicium is integrated in the medicinal fluid container. The non-textual indicia associate the medicinal fluid delivery device with the medicinal fluid container, and hence the medicinal fluid contained therein. The non-textual indicia help to reduce potential error associated with the delivery of medicinal fluids caused by reloading the medicinal fluid delivery device with the wrong medicinal fluid.

The association between a medicinal fluid delivery device and a medicinal fluid container containing a particular medicinal fluid can be helpful in medical environments where a number of different medicinal fluids are administered to a patient. The non-textual indicia helps to reduce the error associated with the delivery of medicinal fluids by allowing the practitioner to quickly and effectively associate a medicinal fluid delivery device with a medicinal fluid container and the medicinal fluid contained therein. In one exemplary embodiment, the medicinal fluid delivery system comprises a medicinal fluid delivery device having a color coded component. Additionally, the medicinal fluid delivery system includes a medicinal fluid container having a color coded component which is associated with the color coded component of the medicinal fluid delivery device. The color coded medicinal fluid delivery device and the medicinal fluid container allow the practitioner to readily identify the medicinal fluid delivery device that is to be utilized with the medicinal fluid container.

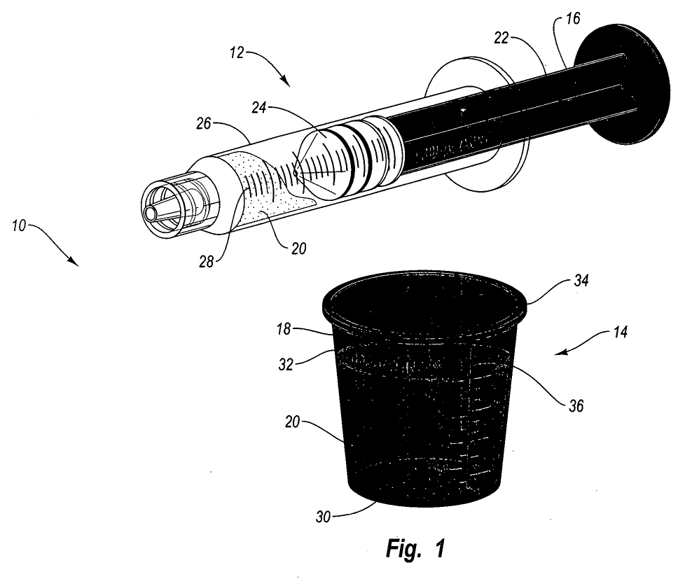

FIG. 1 illustrates a medicinal fluid delivery system 10 according to one embodiment of the present invention. In the illustrated embodiment, medicinal fluid delivery system 10 comprises a medicinal fluid delivery device 12 and an associated medicinal fluid container 14. In the illustrated embodiment, medicinal fluid delivery device 12 comprises a syringe that is configured to facilitate delivery of medicinal fluid 20 to a patient. Medicinal fluid container 14 is configured to receive and hold medicinal fluid 20 therein and to allow removal of medicinal fluid 20 utilizing medicinal fluid delivery device 12. Both, medicinal fluid delivery device 12 includes non-textual indicia 16 and medicinal fluid container 14 includes non-textual indicia, 18. Non-textual indicia 16, 18 indicate that medicinal fluid 20 contained in medicinal fluid container 14 is to be utilized with medicinal fluid delivery device 12. In the illustrated embodiment, non-textual indicia 16, 18 comprise the color of one or more components of medicinal fluid delivery device 12 and medicinal fluid container 14.

In the illustrated embodiment, medicinal fluid delivery device 12 comprises a plunger rod 22, a plunger 24, and an elongate hollow cylindrical barrel 26. Plunger rod 22 is configured to be advanced and retracted by a user to facilitate delivery of medicinal fluid 20 to a patient. Plunger 24 is coupled to one end of plunger rod 22. Plunger 24 is received within elongate hollow cylindrical barrel 26. Plunger 24 C-<n sealingly engages an inner surface of elongate hollow cylindrical barrel 26. In the illustrated embodiment, plunger 24 comprises a resilient material, such as silicone, thermoplastic rubber, other polymers or nonpolymer materials.

Plunger 24 is configured to be advanced within elongate hollow cylindrical barrel 26 by plunger rod 22. Plunger 24 is configured to sealingly engage the inner surface of barrel 26. Elongate hollow cylindrical barrel 26 comprises a uniform cylindrical tube configured to receive medicinal fluid 20 therein. Elongate hollow cylindrical barrel 26 also includes volume indication marks 28. Elongate hollow cylindrical barrel 26 comprises a resilient, semi rigid, or rigid material, such as acrylic, HDPE, or other polymer or nonpolymer material. In one embodiment, barrel 26 has sufficient resilience to withstand deflection under normal use circumstances. In one embodiment, elongate hollow cylindrical barrel 26 is transparent, semitransparent, or translucent to facilitate proper loading and injection of the medicinal fluid utilized with medicinal fluid delivery device 12.

In the illustrated embodiment, the non-textual indicia 16, 18 of medicinal fluid delivery device 12 and medicinal fluid container 14 comprise the color blue. The blue color of medicinal fluid delivery device 12 and medicinal fluid container 14 serve to associate medicinal fluid delivery device 12 with medicinal fluid container 14. The color of medicinal fluid delivery device 12 and medicinal fluid container 14 is an example of non-textual indicia 16, 18 used to associate medicinal fluid delivery device 12 with medicinal fluid container 14 and medicinal fluid 20 contained therein.

During the course of a medical procedure the practitioner fills medicinal fluid container 14 with a predetermined amount of medicinal fluid 20. The color of medicinal fluid container 14 is associated with medicinal fluid 20. For example, in the event the color blue is associated with the medicinal fluid lidocaine, the practitioner can quickly identify that the lidocaine is to be poured into fluid container 14. When the practitioner is ready to inject lidocaine into the patient as a part of the procedure, the practitioner can quickly identify the blue color of plunger rod 22 of medicinal fluid delivery device 12 with the blue color of medicinal fluid container 14. The use of non-textual indicia 16, 18, or in other words the blue color coded components of medicinal fluid delivery device 12 and medicinal fluid container 14, allow the practitioner to make such associations without reading the labels on medicinal fluid delivery device 12 and medicinal fluid container 14. The textual indicia can be utilized as an additional safeguard to ensure that the proper medicament is utilized with the proper medicinal fluid delivery device and the proper medicinal fluid container.

During a medical procedure, in the event that the practitioner administers all or substantially all of medicinal fluid 20 contained in medicinal fluid delivery device 12, the non-textual indicia 16, 18 facilitates reloading of medicinal fluid delivery device 12. During reloading, indicia 16, 18 allow the practitioner to quickly and effectively identify that medicinal fluid delivery device 12 is to be loaded with medicinal fluid 20 from medicinal fluid container 14. The practitioner can quickly and effectively make this association as a result of the color association between non textual indicia 16, 18 of medicinal fluid delivery device 12 and medicinal fluid container 14.

In the illustrated embodiment, plunger rod 22 of medicinal fluid delivery device 12 is colored blue. In one embodiment, the material from which plunger rod 22 is molded or otherwise manufactured is blue. Elongate hollow cylindrical barrel 26 is transparent allowing the user to clearly view the color of plunger rod 22 as well as fluid contained therein. Utilizing plunger rod 22 as the color coded or other non-textual indicia 16 allows the color coded or non-textual indicia 16 to be manufactured as a separate piece, independent from, and thus not linked to, the other components of medicinal fluid delivery device 12. This allows plunger rod 22 to be manufactured in a variety of colors which then can be utilized with other standard components. The plunger rod can be custom ordered to have a particular color and size thus allowing the user to integrate the plunger rod into existing syringe barrels. Additionally, a customer, upon ordering many medicinal fluid delivery systems 10, may desire to have medicinal fluid delivery systems having different colors combinations. As such, the appropriate colored plunger rod 22 can be assembled with an existing elongate hollow cylindrical barrel 26 to provide the desired combination of color coded medicinal fluid delivery devices 12.

As will be appreciated by those skilled in the art, a variety of components of the medicinal fluid delivery device can be color coded or include other non-textual indicia without departing from the scope and spirit of the invention. For example, in one embodiment, the plunger is colored. In another embodiment, the elongate hollow cylindrical barrel includes textual and/or non-textual indicia. In yet another embodiment, the volume indication marks are color coded or include other indicia. In yet another embodiment, both the plunger rod and elongate hollow cylindrical barrel include indicia.

As discussed previously, medicinal fluid container 14 is color coded to associate medicinal fluid delivery device 12 with medicinal fluid container 14. In the illustrated embodiment, the medicinal fluid container 14 includes a base 30, a sidewall 32, and a ring lip 34. Side wall 32 is integrally coupled to base 30. Ring lip 34 is integrally coupled to an opposing end of side wall 32. Medicinal fluid container 14 is configured to receive and hold medicinal fluid 20 while allowing the practitioner to access the medicinal fluid 20 from medicinal fluid container 14 to load medicinal fluid delivery device 12 during a surgical procedure.

Medicinal fluid container 14 further includes volume indication marks 36. Volume indication marks 36 are utilized by a practitioner to determine the volume of medicinal fluid 20 contained in medicinal fluid container 14. Medicinal fluid container 14 comprises a resilient material, semi-rigid, or rigid material, such as HDPE or some other type of plastic sufficient to maintain the integrity and shape of medicinal fluid container 14 during use. In the illustrated embodiment, all of medicinal fluid container 14 is colored. In one embodiment, the material from which medicinal fluid container 14 is molded or otherwise manufactured is blue. In another embodiment, only a portion of the medicinal fluid container includes a color coded component.

As will be appreciated by those skilled in the art, a variety of portions of medicinal fluid container 14 can be color coded or include other non-textual indicium 18 without departing from the scope and spirit of the present invention. For example, in one embodiment only ring lip 34 of medicinal fluid container 14 is colored blue. In another embodiment, only base 30 is colored blue. In yet another embodiment, both base 30 and lip 34 are colored blue. In yet another embodiment, only volume indicator marks 36 are blue.

Further, as will be appreciated by those skilled in the art, a variety of types and configurations of medicinal fluid containers can be utilized without departing from the scope and spirit of the invention. For example, in one embodiment, the medicinal fluid container is a basin. In an alternative embodiment, the medicinal fluid container is a bottle. In yet another embodiment, the medicinal fluid container is a medicine vial. In yet another embodiment, the medicinal fluid container is an infusate bag, such as an IV drip bag. As will be appreciated by those skilled in the art, the terminology medicinal fluid is not limited to fluids having medicinal qualities but can include non-medicinal fluids such as saline, antiseptic treatments, contrast media and other fluids and materials.

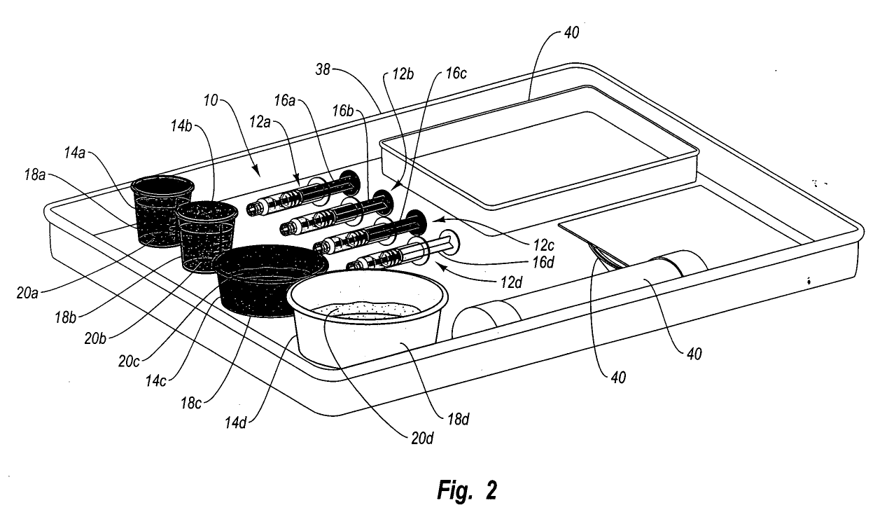

FIG. 2 illustrates a medical tray 38 having one or more medical apparatus 40 placed thereon which are typically utilized during a particular medical procedure. In the illustrated embodiment, medical tray 38 includes a plurality of medicinal fluid delivery devices 12a-d, a plurality of associated medicinal fluid containers 14a-d, and various other medical apparatus 40. Each of the plurality of medical fluid delivery devices 12a-d is utilized with a different medicinal fluid for a different portion of the procedure.

For example, during a diagnostic procedure where one or more catheters are inserted into the body of a patient, medical apparatus 40 include, among other things, one or more diagnostics catheters, guide catheters and diagnostic guide wires. During the diagnostic procedure many medicinal fluids are employed, for example, to relieve pain, treat an area of the patient's body, or flush an inserted catheter. As such, multiple medicinal fluids 20a-d, medicinal fluid delivery devices 12a-d, and medicinal fluid containers 14a-d are required. In the medical environment where multiple medicinal fluids 20a-d, medicinal fluid delivery devices 12a-d, and medicinal fluid containers 14a-d are required, confusion as to which medicinal fluid delivery device contains a particular medicinal fluid may arise. The non-textual indicium 16a-d of medicinal fluid delivery devices 12a-d, respectively, and corresponding indicium 18a-d of medicinal fluid container 20a-d, allow a practitioner to quickly and effectively associate a medicinal fluid delivery device with a medicinal fluid container and the medicinal fluid contained therein.

For example, one medicinal fluid delivery device may be utilized for saline, another may be utilized with a topical anesthetic, while another is utilized with contrast media, while yet another is utilized with a thrombolytic drug. By providing a different colored medicinal fluid delivery device 12 for each medicinal fluid, confusion as to which medicinal fluid delivery device 12 contains which medicinal fluid 20 is minimized. Additionally, confusion as to which associated medicinal fluid container 14 contains the selected medicinal fluid 20 for refilling the medicinal fluid delivery device 12 is minimized.

As will be appreciated by those skilled in the art, a variety of types and configurations of non-textual indicia can be utilized without departing from the scope and spirit of the present invention. For example, a different pattern such as dotted, striped, checkered or the like can be utilized to associate components of the medicinal fluid delivery system. In another embodiment, symbols are utilized to associate components of the medicinal fluid delivery system. In another embodiment, the surface texture of the medicinal fluid delivery system is adapted to associate components of the medicinal fluid delivery system. In one embodiment, the non-textual indicia associate two components of the medicinal fluid delivery system. In another embodiment, the non-textual indicia associate three or more components of the medicinal fluid delivery system. In another embodiment, a plurality of non-textual indicia are utilized together to provide the desired effect. For example, color coding can be utilized with symbols or patterns to associate two or more medical devices.

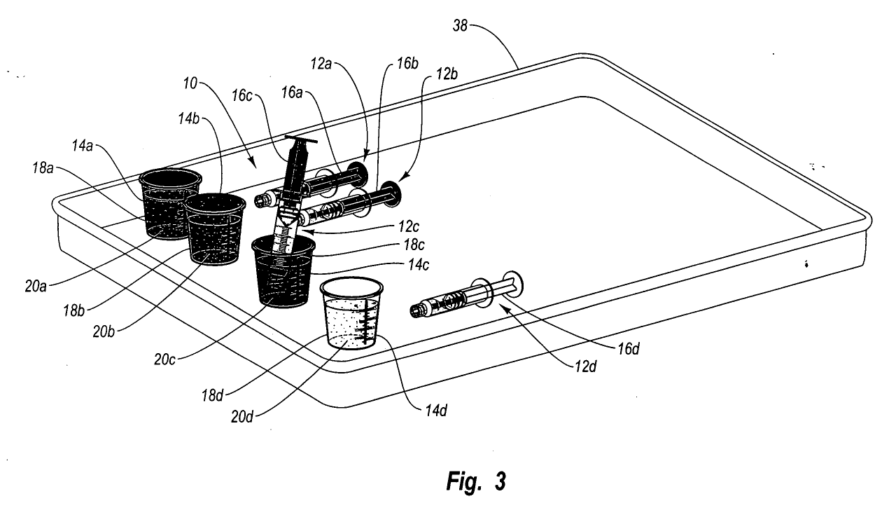

FIG. 3 illustrates operation of the components of medicinal fluid delivery system 10 in a medical environment. As previously discussed, medicinal fluid delivery system 10 can be helpful in medical environments where multiple medicinal delivery devices and medicinal fluids are to be utilized during the course of a procedure. In preparation for a medical procedure, medical tray 38 is prepared with appropriate medical equipment. Multiple medicinal fluid delivery devices 12a-d are utilized in medical procedures in which multiple medicinal fluids are to be administered to the patient or are otherwise utilized during the procedure. During the course of a procedure, one or more medicinal fluid delivery devices 12a-d may be emptied and will need to be reloaded with the appropriate medicinal fluid. For example, during a medical procedure a practitioner utilizes each of medicinal fluid delivery devices 12a-d to deliver medicinal fluids 20a-d contained in medicinal fluid delivery devices 12a-d to the patient.

In the event that the patient requires additional medicinal fluid 20c during the course of the procedure, the practitioner can quickly identify that medicinal fluid delivery device 12c should be utilized. At this point, the practitioner reloads medicinal fluid delivery device 12c with medicinal fluid 20c from medicinal fluid container 14c. The reloading procedure is accomplished effectively and quickly by the practitioner by being able to associate the medicinal fluid delivery device 12c and medicinal fluid container 14c utilizing the color coding of the non-textual indicia 16c, 18c. The practitioner can quickly and effectively make this association because of the corresponding color of medicinal fluid delivery device 12c and medicinal fluid container 14c.

In medical environments where multiple medicinal fluids are needed medicinal fluid containers 14a-d and medicinal fluid delivery devices 12a-d can facilitate safe and effective use of such multiple medicinal fluids. The corresponding indicia 16a-d, 18a-d, facilitate the quick and effective association of medicinal fluid delivery devices 12a-d with associated medicinal fluid containers 14a-d, respectively, and medicinal fluid 20a-d contained therein. The quick and efficient association between medicinal fluid delivery devices 12a-d and medicinal fluid containers 14a-d help to reduce the inadvertent errors and/or association between the incorrect medicinal fluids.

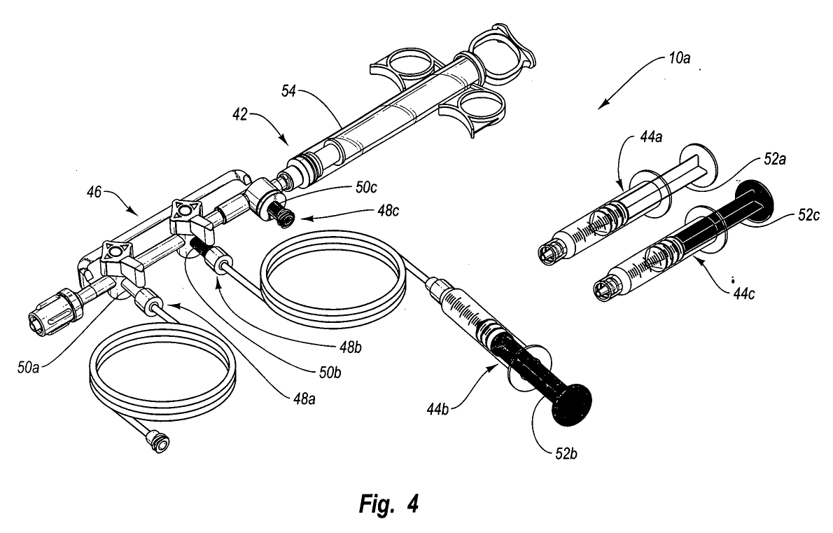

FIG. 4 illustrates an alternative embodiment of a medicinal fluid delivery system 10a according to the present invention. Medicinal fluid delivery system 10a includes a medicinal fluid delivery device 42, first, second and third medicinal fluid containers 44a-c, and non-textual indicia 50a-c, 52a-c. Non-textual indicia 50a-c, 52a-c associate medicinal fluid delivery device 42 with a particular medicinal fluid container 44a-c. Medicinal fluid delivery device 42 comprises a control syringe 54 including a manifold 46 having first, second and third input ports 48a-c. Control syringe 54 is configured to be removably linked to manifold 46 such that control syringe 54 is in fluid communication with manifold 46 and first, second and third input ports 48a-c. The combination of control syringe 54 and manifold 46 facilitates the delivery of one or more medicinal fluids to a patient.

First, second and third input ports 48a-c include non-textual indicia 50a-c integrated respectively therein. Non-textual indicia 50a-c are utilized by a practitioner to associate first, second and third input ports 48a-c with first, second and third medicinal fluid containers 44a-c, respectively. In the illustrated embodiment, medicinal fluid containers 44a-c comprise syringes instead of cups. As will be appreciated by those skilled in the art, medicinal fluid containers can comprise any apparatus utilized to hold a medicinal fluid for an amount of time. First, second and third medicinal fluid containers 44a-c include non-textual indicia 52a-c integrated respectively therein. Non-textual indicia 50a of first input port 48a corresponds with non-textual indicia 52a of first medicinal fluid container 44a. Non-textual indicia 50a of second input port 48a corresponds with non-textual indicia 52a of second medicinal fluid container 44a. Non-textual indicia 50a of third input port 48a corresponds with non-textual indicia 52a of third medicinal fluid container 44a.

Non-textual indicia 50a-c of medicinal fluid delivery device 42 and corresponding non-textual indicia 52a-c of medicinal fluid containers 44a-c serve to associate first, second and third input ports 48a-c to first, second and third medicinal fluid containers 44a-c, respectively, and the medicinal fluids contained therein. The association between non-textual indicia 50a and corresponding indicia 52a allows a practitioner to quickly and effectively associate first input port 48a with first medicinal fluid container 44a. Likewise, non-textual indicia 50b, 50c and corresponding non-textual indicia 52b, 52c, respectively, allow a practitioner to quickly and effectively associate second and third input ports 48b, 48c with second and third medicinal fluid containers 44b, 44c, respectively.

In one embodiment of the present invention, the non-textual indicia 50a-c includes a unique color, and non-textual indicia 52a-c includes a respective corresponding color. For example, the color of first input port 48a corresponds to the color of first medicinal fluid container 44a. The color of second input port 48b corresponds to the color of second medicinal fluid container 44b, but is different from the color of first input port 48a. The color of third input port 48c corresponds to the color of third medicinal fluid container 44c, but is different from both the color of first input port 48a and the color of second input port 48b. The use of color allows a practitioner to quickly and effectively associate an input port with a medicinal fluid container and the medicinal fluid contained therein.

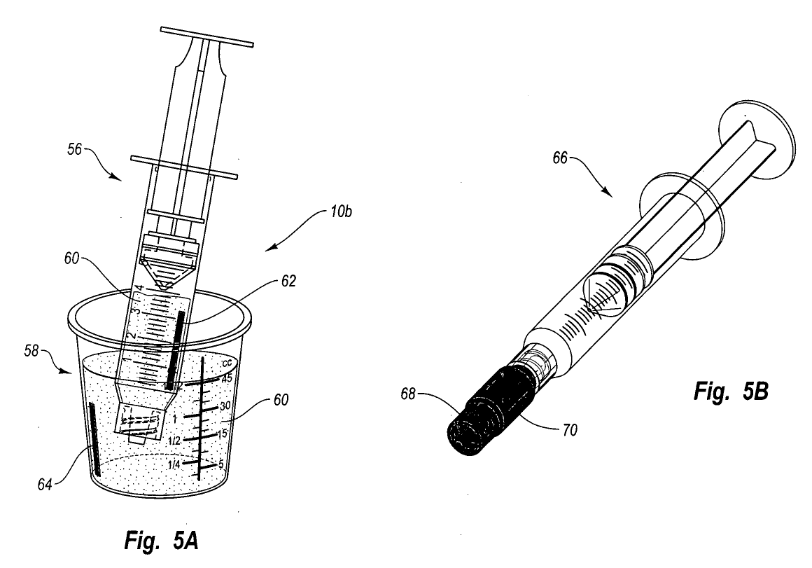

FIG. 5A illustrates an alternative embodiment of a medicinal fluid delivery system 10b of the present invention. Medicinal fluid delivery system 10b includes a medicinal fluid delivery device 56, a medicinal fluid container 58, and chemical indicator strips 62, 64. In the illustrated embodiment, chemical indicator strips 62, 64 comprise non-textual indicia. Chemical indicator strips 62, 64 include an exposed reactant surface. The exposed reactant surface is configured to display a particular color when contacted by a medicinal fluid. For example, when a first medicinal fluid contacts the exposed reactant surface of the chemical indicator strip 62 the exposed reactant surface displays a color corresponding to the medicinal fluid. An indicator strip is one example of a means for indicating material properties of the medicinal fluid.

In one embodiment, chemical indicator strip 62 is embedded in the barrel of medicinal fluid delivery device 56. Chemical indicator strip 64 is integrated into the inner wall of medicinal fluid container 58. The exposed reactant surfaces of chemical indicator strips 62, 64 are configured such that when the exposed reactant surfaces contact medicinal fluid 60 the exposed reactant surface of chemical indicator strip 62 displays a color that corresponds to the color displayed by the exposed reactant surface of other chemical indicator strips exposed to the same medicinal fluid, such as chemical indicator strip 64. The corresponding color displayed by the exposed reactant surface of chemical indicator strips 62, 64 facilitate the association of medicinal fluid delivery device 56 with medicinal fluid container 58 and medicinal fluid 60 contained therein.

FIG. 5B illustrates an alternative medicinal fluid delivery device 66. Medicinal fluid delivery device 66 includes an attachment member 68 having a non-textual indicia 70 removably coupled thereto. Attachment member 68 is coupled to medicinal fluid delivery device 66. In the illustrated embodiment, attachment member 68 comprises a luer coupler having an exposed reactant surface. When attachment member 68 comes in contact with a medicinal fluid, the exposed reactant surface changes to a color indicating the type of medicinal fluid. In the illustrated embodiment, the color displayed by the exposed reactant surface of attachment member 68 comprises the non-textual indicia.

In another embodiment, a colored removable member such as attachment member 68 is provided. The removable member can easily and quickly be added to existing components such as a syringe to indicate to a practitioner the type of medicinal fluid with which it should be utilized. In an alternative embodiment, an attachment member comprising a stop cock is utilized. In one embodiment, the non-textual indicium comprises the color of the material of the attachment member. In an alternative embodiment, a colored component of the medicinal fluid delivery device is utilized with both a chemical indicator strip and textual indicia. For example, a colored component can be utilized in connection with a chemical indicator strip having an exposed reactant surface which displays a color associated with the colored component when contacted by the correct medicinal fluid.

As will be appreciated by those skilled in the art, a variety of types and configurations of means for indicating can be utilized without departing from the scope and spirit of the present invention. For example, in one embodiment the means for indicating comprises a numerical indicator of the properties of the medicinal fluid such as the Ph or other material property of the medicinal fluid. In another embodiment an indicator providing a binary indication as to whether the chemical within the medicinal fluid container, the medicinal fluid delivery device, and/or another component of the medicinal fluid delivery system comprises the desired medicinal fluid. In yet another embodiment, a warning indication is provided when an incorrect medicinal fluid is contained within the medicinal fluid container, the medicinal fluid delivery device, and/or another component of the medicinal fluid delivery system.

As will be appreciated by those skilled in the art, the medicinal fluid delivery devices and medicinal fluid containers can include textual indicia in combination with the non-textual indicia. For example, a medicinal fluid delivery device and a medicinal fluid container can include the name of the medicinal fluid utilized therewith printed on an outer surface the barrel. Alternatively, the textual indicia can be printed on only the medicinal fluid delivery device or the medicinal fluid container, but not both.

The present invention can be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A medicinal fluid delivery system for minimizing error associated with the delivery of medicinal fluids in medical environments where multiple medicinal fluids, medicinal fluid containers and medicinal fluid delivery devices are utilized by practitioners, the system comprising:
a medicinal fluid container configured to hold a medicinal fluid and to allow a medicinal fluid to be selectively removed therefrom, the medicinal fluid container having a first non-textual indicium integrated in the medicinal fluid container; and
a medicinal fluid delivery device configured to facilitate delivery of at least one medicinal fluid to a patient, wherein the medicinal fluid delivery device includes a second non-textual indicium that corresponds with the first non-textual indicium to associate the medicinal fluid delivery device with the medicinal fluid container, such that a practitioner can quickly and effectively identify the medicinal fluid container associated with the medicinal fluid delivery device.

2. The system as recited in claim 1, wherein the first non-textual indicium is a first color and the second non-textual indicium is a second color corresponding with the first color.

3. The system as recited in claim 2, wherein the medicinal fluid delivery device comprises a medical syringe.

4. The system as recited in claim 3, wherein the medical syringe comprises a plunger rod, wherein the second color is the color of the plunger rod.

5. The system as recited in claim 2, wherein the medicinal fluid delivery device comprises a manifold configured to facilitate delivery of one or more medicinal fluids to a patient, wherein the manifold comprises a first inlet port, wherein the second non-textual indicium is integrated in a first inlet port.

6. The system as recited in claim 5, further comprising a second medicinal fluid container having a third non-textual indicium integrated in the second medicinal fluid container, and wherein the manifold further comprises a second inlet port and a fourth non-textual indicium integrated in the second inlet port, wherein the fourth non-textual indicium corresponds to the third non-textual indicium, such that the first inlet port is associated with the first medicinal fluid container and the second inlet portion is associated with the second medicinal fluid container.

7. The system as recited in claim 6, wherein the first medicinal fluid container comprises a medical syringe.

8. The system as recited in claim 1, wherein the medicinal fluid container comprises a first chemical indicator strip having an exposed reactant surface, and wherein the medicinal fluid delivery device comprises a second chemical indicator strip having an exposed reactant surface.

9. The system as recited in claim 8, wherein the exposed reactant surfaces are configured to display corresponding colors when the exposed reactant surfaces are contacted by the same medicinal fluid, such that the medicinal fluid delivery device is associated with the medicinal fluid container.

10. The system as recited in claim 9, wherein the medicinal fluid delivery device is a medical syringe and the second chemical indicator strip is integrated in the medical syringe.

11. The system as recited in claim 9, wherein the medicinal fluid delivery device comprises a manifold having at least one input port.

12. The system as recited in claim 11, wherein the medicinal fluid container comprises a medical syringe.

13. The system as recited in claim 12, wherein the first chemical indicator strip is integrated in the medical syringe, and wherein the second chemical indicator strip is integrated in the first input port.

14. A medicinal fluid delivery system for minimizing error associated with the delivery of medicinal fluids in medical environments where multiple medicinal fluids, medicinal fluid containers and medicinal fluid delivery devices are utilized by a practitioner, the system comprising:
a medicinal fluid delivery device configured to receive and deliver medicinal fluids, the device comprising:
an elongate cylindrical tube adapted to receive a medicinal fluid therein; and
a plunger associated with the elongate cylindrical tube and configured to facilitate the delivery of a medicinal fluid to a patient,
wherein the medicinal fluid delivery device includes a non-textual indicium integrated in the medicinal fluid delivery device to associate the medicinal fluid delivery device with a medicinal fluid; and
a medicinal fluid container configured to hold a medicinal fluid therein and to allow a medicinal fluid to be selectively removed therefrom, wherein the medicinal fluid container includes a non-textual indicium integrated in the medicinal fluid container to associate the medicinal fluid container with the medicinal fluid delivery device, such that a practitioner can quickly and effectively identify the medicinal fluid container associated with the medicinal fluid delivery device.

15. The system as recited in claim 14, wherein the non-textual indicium of the medicinal fluid container is a first color.

16. The system as recited in claim 15, wherein the non-textual indicium of the medicinal fluid delivery device is a second color, wherein the second color corresponds with the first color.

17. The system as recited in claim 16, wherein the first color is the color of the medicinal fluid container.

18. The system as recited in claim 16, wherein the second color is the color of the plunger rod.

19. The system as recited in claim 16, wherein the medicinal fluid container comprises a first chemical indicator strip having an exposed reactant surface, and wherein the medicinal fluid delivery device comprises a second chemical indicator strip having an exposed reactant surface.

20. The system as recited in claim 19, wherein the exposed reactant surfaces are configured to display corresponding colors when the exposed reactant surfaces are contacted by the same medicinal fluid, such that the medicinal fluid delivery device is associated with the medicinal fluid container.

21. The system as recited in claim 20, wherein the first color is the color displayed by the exposed reactant surface of the first chemical indicator strip.

22. The system as recited in claim 20, wherein the second color is the color displayed by the exposed reactant surface of the second chemical indicator strip.

23. A method of utilizing color-coded medicinal fluid delivery components for minimizing error associated with the delivery of medicinal fluids in medical environments where multiple medicinal fluids, medicinal fluid containers and medicinal fluid delivery devices are utilized by practitioners, the method comprising:
placing medicinal fluid in a medicinal fluid container configured to hold the medicinal fluid therein and to allow the medicinal fluid to be selectively removed therefrom, wherein the medicinal fluid container includes a color; and
filling a medical delivery device with medicinal fluid from the medicinal fluid container, wherein the medical delivery device includes a color that corresponds to the color of the medicinal fluid container, such that a practitioner can quickly and effectively identify the medicinal fluid container corresponding with the medical delivery device; and
delivering the medicinal fluid from the medical syringe to the patient.

24. The system as recited in claim 23, wherein the color of the medicinal fluid container and the medical delivery device comprising the same color.

25. The system as recited in claim 23, wherein the medical delivery device comprises a syringe.

26. The system as recited in claim 25, wherein the medicinal fluid container comprises a first chemical indicator strip having an exposed reactant surface, and wherein the medical syringe comprises a second chemical indicator strip having an exposed reactant surface.

27. The system as recited in claim 26, wherein the exposed reactant surfaces are configured to display corresponding colors when the exposed reactant surfaces are contacted by the same medicinal fluid, such that the medical syringe is associated with the medicinal fluid container.

28. The system as recited in claim 27, wherein the first color is the color displayed by the exposed reactant surface of the first chemical indicator strip.

29. The system as recited in claim 27, wherein the second color is the color displayed by the exposed reactant surface of the second chemical indicator strip.

30. In a medical environment in which medicinal fluids are to be administered, a method of delivering a medicinal fluid, comprising:
providing a container for holding a medicinal fluid therein, wherein the container comprises non-textual indicia integrated in the container;
providing a medical syringe that is configured to receive a medicinal fluid from the container and deliver the medicinal fluid to a patient, wherein the medical syringe includes non-textual indicia that corresponds to the non-textual indicia of the container, allowing the practitioner to quickly and effectively identify the medicinal fluid container corresponding with the medical syringe; and
loading the medical syringe with a medicinal fluid from the corresponding medicinal fluid container.

31. The method as recited in claim 30, wherein the first non-textual indicium is a first color.

32. The method as recited in claim 31, wherein the second non-textual indicium is a second color, wherein the second color corresponds with the first color.

33. The method as recited in claim 32, wherein the first color is the color of the medicinal fluid container.

34. The method as recited in claim 33, wherein the second color is the color of the medical syringe.

35. The method as recited in claim 34, further comprising the step of reloading the medical syringe with the medicinal fluid from the corresponding medicinal fluid container.

36. The method as recited in claim 30, wherein the medicinal fluid container comprises a first chemical indicator strip having an exposed reactant surface, and wherein the medical syringe comprises a second chemical indicator strip having an exposed reactant surface.

37. The method as recited in claim 36, wherein the exposed reactant surfaces are configured to display corresponding colors when the exposed reactant surfaces are contacted by the same medicinal fluid, such that the medical syringe is associated with the medicinal fluid container.

38. The method as recited in claim 37, wherein the first non-textual indicium is the color displayed by the exposed reactant surface of the first chemical indicator strip.

39. The method as recited in claim 38, wherein the second non-textual indicium is the color displayed by the exposed reactant surface of the second chemical indicator strip.

40. The method as recited in claim 39, further comprising the step of reloading the medical syringe with the medicinal fluid contained in the corresponding medicinal fluid container.
